(19) United States
(12) Patent Application Publication
(10) Pub. No.: US 2024/0398850 A1
(43) Pub. Date: Dec. 5, 2024

(54) VISCOSUPPLEMENT COMPOSITIONS

(71) Applicant: THE UNIVERSITY OF BRITISH COLUMBIA, Vancouver (CA)

(72) Inventors: Dana GRECOV, Port Coquitlam (CA); Akshai BOSE, Vancouver (CA); Behzad ZAKANI, Vancouver (CA)

(21) Appl. No.: 18/679,820

(22) Filed: May 31, 2024

Related U.S. Application Data
(60) Provisional application No. 63/469,933, filed on May 31, 2023.

(51) Int. Cl. A61K 31/728; A61K 9/00; A61K 9/08; A61K 31/717

(52) U.S. Cl. CPC A61K 31/728; A61K 9/0019; A61K 9/08; A61K 31/717

(57) ABSTRACT

The present disclosure relates to viscosupplements and uses thereof, for example, in viscosupplementation of a mammalian joint and/or in methods of treating osteoarthritis. For example, the viscosupplements can comprise hyaluronic acid and/or a derivative thereof and crystalline nanocellulose, in a physiologically acceptable buffered saline.

### CROSS-REFERENCE TO RELATED APPLICATIONS

The present application claims the benefit of priority from co-pending U.S. provisional application No. 63/469,933 filed on May 31, 2023, the contents of which are incorporated herein by reference in their entirety.

### FIELD

The present disclosure relates to viscosupplements comprising, for example, hyaluronic acid and/or a derivative thereof and crystalline nanocellulose, as well as uses thereof, for example, in viscosupplementation of a mammalian joint and/or in methods of treating osteoarthritis.

### BACKGROUND

The synovial joint is a freely movable joint in the human skeleton, and includes a synovium that encapsulates synovial fluid (SF). The SF helps to provide lubrication and the transfer of nutrients. The presence of hyaluronic acid (HA) in the SF provides exceptional lubrication properties. Osteoarthritis (OA) is a commonly occurring degenerative disease characterized by the breakdown of cartilage in joints, and where the SF loses its lubrication properties due to the degradation of HA. Symptoms can include joint pain, joint stiffness after prolonged inactivity, and limited movement of joints with the disease progression. OA is prevalent in many joints of the body but occurs frequently in the knee and hip joints. Common treatments of OA involve intra-articular injections of steroids or viscosupplements into the joint, and/or joint replacement surgery.

Viscosupplements reduce pain due to direct bone-to-bone contact or cartilage-to-cartilage contact and increase joint mobility in OA patients. Viscosupplementation often occurs using a single, or multiple (e.g., three or five) injection cycle. However, the therapeutic effects of viscosupplementation typically do not last for long periods of time and frequent repeated treatments are often required. The vast majority of viscosupplements are based on HA and/or its derivatives. It has been observed that the injected HA degrades in the joint over time, leading to a loss of the benefit of viscosupplementation. Therefore, the stability and the lubrication performance are important properties of HA-based viscosupplements in the treatment of osteoarthritis.

### SUMMARY

Reported herein are viscosupplements and methods of their use, designed to overcome at least some of the disadvantages of current solutions. For example, we have found that viscosupplements of the disclosed compositions can, for example, provide for reduced oxidative breakdown of the HLA species and improved lubrication properties.

Accordingly, the present disclosure includes a method of viscosupplementation of the synovial fluid of a joint in a mammalian subject, the method comprising.
intra-articularly injecting into the joint, a viscosupplement comprising:
   hyaluronic acid and/or a derivative thereof; and
   crystalline nanocellulose,
in a physiologically acceptable buffered saline.

In an embodiment, the viscosupplementation is for treatment of arthritis. In another embodiment, the arthritis is osteoarthritis.

In an embodiment, the crystalline nanocellulose has been prepared by a method comprising sulphuric acid hydrolysis. In another embodiment, the crystalline nanocellulose is present at a concentration of from 0.1 wt. % to 3 wt. %, based on the total weight of the viscosupplement.

In an embodiment, the hyaluronic acid and/or derivative thereof comprises a sodium salt of hyaluronic acid. In another embodiment, the hyaluronic acid is from a bacterial source.

In an embodiment, the physiologically acceptable buffered saline is a phosphate-buffered saline.

In an embodiment, the mammalian subject is a human.

In an embodiment, the mammalian subject is a dog or a horse.

In an embodiment, the number and/or frequency of injections is lower than the number and/or frequency of injections with a corresponding viscosupplement comprising the hyaluronic acid and/or a derivative thereof in the physiologically acceptable buffered saline but devoid of the crystalline nanocellulose.

The present disclosure also includes a viscosupplement comprising:
hyaluronic acid and/or a derivative thereof; and
crystalline nanocellulose,
in a buffer solution.

The present disclosure also includes a method of treating osteoarthritis in a joint of a mammalian subject suffering from osteoarthritis in the joint, the method comprising:
intra-articularly injecting into the joint, a viscosupplement comprising:
   hyaluronic acid and/or a derivative thereof; and
   crystalline nanocellulose,
in a physiologically acceptable buffered saline.

In an embodiment, the crystalline nanocellulose has been prepared by a method comprising sulphuric acid hydrolysis and/or wherein the crystalline nanocellulose is present at a concentration of from 0.1 wt. % to 3 wt. %, based on the total weight of the viscosupplement.

In an embodiment, the hyaluronic acid and/or derivative thereof comprises a sodium salt of hyaluronic acid. In another embodiment, the hyaluronic acid is from a bacterial source.

In an embodiment, the physiologically acceptable buffered saline is a phosphate-buffered saline.

In an embodiment, the mammalian subject is a human.

In an embodiment, the mammalian subject is a dog or a horse.

In an embodiment, the number and/or frequency of injections is lower than the number and/or frequency of injections with a corresponding viscosupplement comprising the hyaluronic acid and/or a derivative thereof in the physiologically acceptable buffered saline but devoid of the crystalline nanocellulose.

Other features and advantages of the present disclosure will become apparent from the following detailed description. It should be understood, however, that the detailed description and the specific examples, while indicating embodiments of the disclosure, are given by way of illustration only and the scope of the claims should not be limited by these embodiments, but should rather be given the broadest interpretation consistent with the description as a whole.

### BRIEF DESCRIPTION OF THE DRAWINGS

The embodiments of the disclosure will now be described in greater detail with reference to the attached drawings, in which:

FIG. 1 shows a geometrical representation of the wear area calculation.

FIG. 2 shows polarizing optical microscope (POM) images (right column) and photomicrographs (left column) of different samples of Table 1 (from top to bottom in each column: HA, HA+0.125CNC, HA+0.25CNC, HA+0.375CNC and HA+0.5CNC) under 500× optical zoom. Scale bar in each image shows 100 μm.

FIG. 3 is a schematic showing possible intermolecular and intramolecular hydrogen bonding occurring between and within the moieties of CNC and HA.

FIG. 4 is a plot showing steady shear viscosity of the samples HA+0.125CNC, HA+0.25CNC, HA+0.375CNC and HA+0.5CNC in comparison to HA at room temperature.

FIG. 5 is a plot showing shear stress vs. shear rate using the experimental data from steady shear viscometry and the Herschel-Bulkley model for samples HA+0.125CNC, HA+0.25CNC, HA+0.375CNC and HA+0.5CNC in comparison to HA.

FIG. 6 is a plot showing coefficient of friction (COF) of different samples (HA+0.125CNC, HA+0.25CNC, HA+0.375CNC and HA+0.5CNC) in comparison to HA under 5 N and 10 N (*) loads for steel-tribo pairs with 150 mm/min speed.

FIG. 7 is a plot showing variation of COF over time for HA+0.125CNC, HA+0.25CNC, HA+0.375CNC and HA+0.5CNC in comparison to HA for a 5 N load and a speed of 150 mm/min using a ceramic-tribo pair.

FIG. 8 is a plot showing variation of COF over time for HA+0.125CNC, HA+0.25CNC, HA+0.375CNC and HA+0.5CNC in comparison to HA for a 10 N load and a speed of 150 mm/min with a ceramic-tribo pair.

FIG. 9 is a plot showing comparison of COF for HA+0.125CNC, HA+0.25CNC, HA+0.375CNC and HA+0.5CNC to HA using an alumina ceramic-tribo pair under loading conditions of 5 N and 10 N (*) and at a speed of 150 mm/min.

FIG. 10 is a plot showing variation of COF over time for HA+0.125CNC, HA+0.25CNC, HA+0.375CNC and HA+0.5CNC in comparison to HA for a 5 N load and a 150 mm/min speed for a PDMS-tribo pair.

FIG. 11. is a plot showing variation of COF over time for HA+0.125CNC, HA+0.25CNC, HA+0.375CNC and HA+0.5CNC in comparison to HA for a 2 N load and a 75 mm/min speed for the PDMS-tribo pair.

FIG. 12. is a plot showing COF under different loads and speed conditions [5 N and 150 mm/min or 2 N and 75 mm/min (*)] using the PDMS-tribo pair for HA+0.125CNC, HA+0.25CNC, HA+0.375CNC and HA+0.5CNC in comparison to HA.

FIG. 13 shows scanning electron micrograph (SEM) images of steel (left column), ceramic (center column), and PDMS (right column) plates after the pin on disc test conducted under a 5 N load and a speed of 150 mm/min for, from top to bottom: HA, HA+0.25CNC and HA+0.5CNC. In each case, the main image is less magnified (with a 300 μm scale for ceramic and PDMS and a 50 μm scale for steel) and the image at the top-right corner of the main image is higher magnification (with a 50 μm scale for ceramic and PDMS and a 10 μm scale for steel).

FIG. 14 shows 3D images of the wear scars on steel (left column), ceramic (center column, and PDMS (right column) plates after the pin on disc test run under a 5 N load and a speed of 150 mm/min for three samples, from top to bottom: HA, HA+0.25CNC and HA+0.5CNC (in color images, the color scale ranges from −20 μm to 20 μm).

FIG. 15 shows roughness profiles of the steel plates after the pin on disc test at a 150 mm/min speed and at two different loads of 5 N (*) and 10 N (**) for five different samples: HA (upper plot), HA+0.125CNC (middle left plot), HA+0.25CNC (middle right plot), HA+0.375CNC (lower left plot) and HA+0.5CNC (lower right plot).

FIG. 16 shows plots of RMS values of roughness (upper plot); wear depths (middle plot); and wear rates (lower plot) for the steel plates from the tribology test under a 10 N (*) and a 5 N load and at a 150 mm/min speed for, from left to right in each plot: HA, HA+0.125CNC, HA+0.25CNC, HA+0.375CNC and HA+0.5CNC.

FIG. 17 shows roughness profiles of the wear scars for ceramic plates after the tribology test at a 150 mm/min speed and for two different loads of 5 N (*) and 10 N (**) for five different samples: HA (upper plot), HA+0.125CNC (middle left plot), HA+0.25CNC (middle right plot), HA+0.375CNC (lower left plot) and HA+0.5CNC (lower right plot).

FIG. 18 shows plots of RMS values of roughness (upper plot); wear depths (middle plot); and wear rates (lower plot) for ceramic plates after the tribology test with different samples under 10 N (*) and 5 N loads and at a 150 mm/min speed for, from left to right in each plot: HA, HA+0.125CNC, HA+0.25CNC, HA+0.375CNC and HA+0.5CNC.

FIG. 19 shows roughness profiles of the wear scars on the PDMS plates after the tribology test at a 5 N load and a 150 mm/min speed (**); and at a 2 N load and a 75 mm/min speed (*), for five different samples: HA (upper plot), HA+0.125CNC (middle left plot), HA+0.25CNC (middle right plot), HA+0.375CNC (lower left plot) and HA+0.5CNC (lower ight plot).

FIG. 20 shows RMS values of roughness (upper plot); wear depths (middle plot); and wear rate (lower plot) for the PDMS plates after the tribology tests with all samples under different conditions [5 N load and 150 mm/min speed (*); or 2 N load and 75 mm/min speed] for, from left to right in each plot: HA, HA+0.125CNC, HA+0.25CNC, HA+0.375CNC and HA+0.5CNC.

FIG. 21 is a plot showing variation in viscosity over time after adding oxidizing agents of a Weissberger's reaction for HA+0.125CNC, HA+0.25CNC, HA+0.375CNC and HA+0.5CNC in comparison to HA.

FIG. 22 is a plot showing percentage change in viscosity over time after adding the oxidizing agents of a Weissberger's reaction for HA+0.125CNC, HA+0.25CNC, HA+0.375CNC and HA+0.5CNC in comparison to HA.

FIG. 23 shows polarizing optical microscopy images of samples with 0.75 to 7% CNC (from top to bottom on first page of FIG. 23: HA+0.75CNC, HA+1CNC, HA+2CNC and HA+3CNC; and from top to bottom on second page of FIG. 23: HA+4CNC, HA+5CNC, HA+6CNC and HA+7CNC) at two different scales 500 μm (left columns) and 50 μm (right columns).

FIG. 24 is a plot showing steady shear viscosity of samples HA+1CNC, HA+2CNC, HA+3CNC, HA+4CNC and HA+5CNC in comparison to a commercial viscosupplement and the commercial viscosupplement+0.25CNC.

FIG. 25 is a plot showing variation of viscosity over time of the samples (without oxidizing agent) for HA+1CNC, HA+2CNC, HA+3CNC, HA+4CNC and HA+5CNC in comparison to a commercial viscosupplement and the commercial viscosupplement+0.25CNC.

FIG. 26 is a plot showing variation of viscosity over time after adding oxidizing agent into the sample for HA+1CNC, HA+2CNC, HA+3CNC, HA+4CNC and HA+5CNC in comparison to a commercial viscosupplement and the commercial viscosupplement+0.25CNC.

FIG. 27 is a plot showing percentage change in viscosity over time of selected samples with various concentrations of CNC: HA+1CNC, HA+2CNC, HA+3CNC, HA+4CNC and HA+5CNC in comparison to a commercial viscosupplement and the commercial viscosupplement+0.25CNC.

FIG. 28 is a plot showing variation of COF overtime for a 5 N load and a 150 mm/min speed for the steel-tribo pairs for HA+1CNC, HA+2CNC, HA+3CNC and HA+4CNC.

FIG. 29 is a plot showing variation of COF over time for a 10 N load and a 150 mm/min speed for the steel-tribo pairs for HA+1CNC, HA+2CNC, HA+3CNC and HA+4CNC.

FIG. 30 is a plot showing comparison of coefficient of friction of different samples at two different loads, 5 N and 10 N (*) and 150 mm/min speed when the pin on disc test was carried out using steel friction pair for HA+1CNC, HA+2CNC, HA+3CNC and HA+4CNC.

FIG. 31 shows 3D profile of wear tract generated on steel plate after the pin on disc test was run for 1 hour with different samples, from top to bottom. HA, HA+1CNC, HA+2CNC, HA+3CNC and HA+4CNC at a load of 5 N (left) and a load of 10 N (right).

FIG. 32 shows roughness profiles of the steel plates after the pin on disc test at a 150 mm/min speed and 5 N load and different samples: HA+1CNC, HA+2CNC, HA+3CNC and HA+4CNC in comparison to HA.

FIG. 33 shows roughness profiles of the steel plates after the pin on disc test at a 150 mm/min speed and 10 N load and different samples: HA+1CNC, HA+2CNC, HA+3CNC and HA+4CNC in comparison to HA.

FIG. 34 shows SEM images of steel plates after the pin on disc test conducted under 5 N (left column) and 10 N (right column) loads and a speed of 150 mm/min for, from top to bottom. HA, HA+1CNC, HA+2CNC, HA+3CNC and HA+4CNC. In each case, the main image is less magnified (with a 100 μm scale) and the image at the top-left corner of main image is higher magnification (with a 50 μm scale).

FIG. 35 is a plot showing steady-shear viscometry of HA/CNC suspensions at different HA concentrations: 2HA+2CNC, 3HA+2CNC and 4HA+2CNC.

FIG. 36 is a plot showing percentage change in viscosity 1.5 hours after the addition of oxidizing agent to 4HA+2CNC, 3HA+2CNC, 2HA+2CNC and 0.5HA+2CNC.

FIG. 37 is a plot showing COF of samples using steel friction pair under 5 N load and 150 mm/min speed for 4HA+2CNC, 3HA+2CNC, 2HA+2CNC and 0.5HA+2CNC.

FIG. 38 is a plot showing COF of samples using steel friction pair under 10 N load and 150 mm/min speed for 4HA+2CNC, 3HA+2CNC, 2HA+2CNC and 0.5HA+2CNC.

FIG. 39 is a plot showing COF of samples using PDMS friction pair under 2 N load and 150 mm/min speed for 0.5HA+2CNC, 2HA+2CNC, 3HA+2CNC and 4HA+2CNC.

FIG. 40 is a plot showing COF of samples using steel friction pair under 5 N load and 150 mm/min speed for 0.5HA+2CNC, 2HA+2CNC, 3HA+2CNC and 4HA+2CNC.

### DETAILED DESCRIPTION

### I. Definitions

Unless otherwise indicated, the definitions and embodiments described in this and other sections are intended to be applicable to all embodiments and aspects of the disclosure herein described for which they would be understood to be suitable by a person skilled in the art.

As used herein, the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process/method steps.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

As used in this disclosure, the singular forms “a”, “an” and “the” include plural references unless the content clearly dictates otherwise.

The term “and/or” as used herein means that the listed items are present, or used, individually or in combination. In effect, this term means that “at least one of” or “one or more” of the listed items is used or present.

The term “physiologically acceptable” means compatible with the treatment of mammalian subjects such as humans.

The term “suitable” as used herein means that the selection of the particular compound, material and/or conditions would depend on the specific manipulations to be performed, and/or the identity of the compound(s) and/or material(s) to be transformed, but the selection would be well within the skill of a person skilled in the art.

### II. Viscosupplements and Uses Thereof

The present disclosure includes a method of viscosupplementation of the synovial fluid of a joint in a mammalian subject, the method comprising:
intra-articularly injecting into the joint, a viscosupplement comprising:
   hyaluronic acid and/or a derivative thereof; and
   crystalline nanocellulose,
in a physiologically acceptable buffered saline.

In an embodiment, the viscosupplementation is for treatment of arthritis. In another embodiment, the arthritis is osteoarthritis.

The present disclosure also includes a method of treating osteoarthritis in a joint of a mammalian subject suffering from osteoarthritis in the joint, the method comprising:
intra-articularly injecting into the joint, a viscosupplement comprising:
   hyaluronic acid and/or a derivative thereof; and
   crystalline nanocellulose,
in a physiologically acceptable buffered saline.

The crystalline nanocellulose (CNC) may be a material selected from the family of materials commonly referred to as crystalline nanocellulose or cellulose nanocrystals. The CNC may be present in a single form, or a combination of different CNCs. The CNC can be present as one form, or a combination of more than one form of CNC or CNC derivative. Accordingly, a person skilled in the art would readily appreciate that the crystalline nanocellulose can be any suitable crystalline nanocellulose or mixture thereof and/or a derivative thereof, the selection of which can be made by a person skilled in the art. For example, the skilled person would appreciate that the CNC can be present as a mixture of any suitable combination of the CNC embodiments described herein. In an embodiment, the CNC is present as one form of CNC or CNC derivative. In another embodiment, the CNC is present as a mixture of more than one form of CNCs or CNC derivatives. A person skilled in the art would also readily appreciate that the term “crystalline nanocellulose” and the like is generally used to refer to crystals of cellulose in which all dimensions are in the nanometer size range. However, a person skilled in the art would also readily appreciate that the term “crystalline nanocellulose” and the like as used herein may also include materials with suitable minor amounts of material outside of that form. In an embodiment, the CNC has an average width of less than 30 nm. In another embodiment, the CNC has an average width of less than 20 nm. In a further embodiment, the CNC has an average width of less than 10 nm. In another embodiment, the CNC has an average size of 3-6 nm. CNCs are typically prepared by acid hydrolysis of cellulose-based compounds (for example from one or more polymorphs of cellulose such as cellulose I, cellulose II, cellulose III and cellulose IV), most often using carboxylic acid or sulfuric acid. Accordingly, in an embodiment, the CNC is prepared from Cellulose I. In another embodiment, the CNC is prepared from Cellulose II. In a further embodiment, the CNC is prepared from Cellulose III. In another embodiment, the CNC is prepared from Cellulose IV. However, a person skilled in the art would readily appreciate that other suitable methods for preparation of crystalline nanocellulose may be used and that the source of cellulose can be any suitable source or combination thereof. Suitable sources of cellulose are well known in the art and include, for example, suitable plant sources such as wood, cotton, and bast fibers (e.g., flax, hemp or ramie) as well as suitable algal sources. The CNC may be carboxylated CNC. The CNC may be sulphonated CNC. Accordingly, in an embodiment, the CNC is carboxylic acid hydrolyzed CNC. In another embodiment, the CNC is sulfuric acid hydrolyzed CNC. In an embodiment, the crystalline nanocellulose has been prepared by a method comprising sulphuric acid hydrolysis. The CNC may be in a form that has been functionalized, through, for example, surface modification chemistry. Such modification chemistry may include but is not limited to acetylation, TEMPO-mediated oxidation, cationic hydrophobic and/or hydrophilic modifications, via grafting of polymers (for example polyacrylic acid) and surface alkylation. The concentration of the crystalline nanocellulose in the viscosupplement can be any suitable concentration, the selection of which can be readily made by a person skilled in the art having regard to the present disclosure. In an embodiment, the CNC is at a concentration of between 0.005% and 7% (w/w). In another embodiment, the CNC is at a concentration of between 0.01% and 10%. In a further embodiment, the CNC is at a concentration of between 1% and 5%. In another embodiment, the CNC is at a concentration of between 0.1% and 3%. Where reference is made to the concentration of CNC, this refers to the total concentration of the CNC species, by weight, to the total weight of the viscosupplement or viscoelastic solution. For example, in an embodiment, the crystalline nanocellulose is present at a concentration of from 0.1 wt. % to 3 wt. %, based on the total weight of the viscosupplement.

The hyaluronic acid species (i.e., the hyaluronic acid and/or derivative thereof) is present in one or a combination of more than one form of a hyaluronic acid species (HLAS), which includes species which are derivatives of hyaluronic acids. Some examples of such species include hyaluronic acid (hyaluronan), sodium hyaluronate and hylans, which the skilled person would readily appreciate includes hyaluronans crosslinked with formaldehyde (Hylan-A) or with divinylsulfone (Hylan-B. Other examples include other HLAS that are cross-linked or gels formed from HLAS. Accordingly, a person skilled in the art would readily appreciate that the hyaluronic acid and/or derivative thereof can be any suitable hyaluronic acid and/or derivative thereof or mixture thereof, the selection of which can be made by a person skilled in the art. For example, the skilled person would appreciate that the HLAS can be present as a mixture of any suitable combination of the HLAS embodiments described herein. In an embodiment, the HLAS is a salt of a hyaluronic acid or hyaluronic derivative species. In another embodiment, the HLAS is at least partially crosslinked. In an alternative embodiment, the HLAS is not crosslinked. In an embodiment, the HLAS comprises a hyaluronan species. In an embodiment, the HLAS is a hyaluronan or a derivative thereof. In an embodiment, the HLAS comprises a hylan species. In another embodiment, the HLAS is a hylan or mixture of hylans. In another embodiment, the HLAS is a gel. In an embodiment, the hyaluronic acid and/or derivative thereof comprises a sodium salt of hyaluronic acid. In an embodiment, the HLAS has a MW of 0.1 to 10 MDa. In another embodiment, the HLAS has a MW of less than about 6 MDa. In another embodiment, the HLAS has a MW of 2 to 3 MDa. In a further embodiment, the HLAS has a MW of 1.5 to 1.8 MDa. The HLAS may be synthetically produced, biosynthesized from, for example, fermentation processes, or derived from naturally occurring sources such as rooster comb. However, a person skilled in the art would also readily appreciate that other suitable methods of preparation and/or sources of the hyaluronic acid and/or derivative thereof may be used. For example, in another embodiment, the hyaluronic acid is from a bacterial source e.g., Streptooccis equi. HLAS may also include other species from the glycosaminoglycan family, and derivatives thereof, such as polysulphated glycosaminoglycans. The concentration of the hyaluronic acid and/or derivative thereof in the viscosupplement can be any suitable concentration, the selection of which can be readily made by a person skilled in the art having regard to the present disclosure. In an embodiment, the HLAS is at a concentration of 0.1-100 mg/mL. In another embodiment, the HLAS is at a concentration of 1-50 mg/mL. In a further embodiment, the HLAS is at a concentration of 5-30 mg/mL. In another embodiment, the HLAS is at a concentration of 0.5-4 mg/mL. In an embodiment, the HLAS is at a concentration of 1-2 mg/mL. Where reference is made to the concentration of HLAS, this refers to the total HLAS concentration, by weight, per mL of the viscosupplement or viscoelastic solution.

In embodiments of the present disclosure in which the viscosupplements of the present disclosure are for use in a mammalian subject, a person skilled in the art would readily appreciate that such viscosupplements are desirably physiologically acceptable. The physiologically acceptable buffered saline would accordingly have a pH and composition compatible with the treatment of mammalian subjects such as humans and this could be readily selected by a person skilled in the art. In an embodiment, the physiologically acceptable buffered saline is a phosphate buffered saline (PBS). In an embodiment, the pH of the viscosupplement is 7.2±0.5. In another embodiment, the pH of the viscosupplement is 7.4+/−0.5.

In an embodiment, the mammalian subject is a human. In another embodiment, the mammalian subject is a non-human mammal. In another embodiment, the mammalian subject is a dog or a horse. In an embodiment, the mammalian subject is a dog. In an embodiment, the mammalian subject is a horse. The joint can be any joint suitable for viscosupplementation. In an embodiment, the joint is a knee, hip, ankle, toe, shoulder, elbow, wrist or finger. In an embodiment, the joint is a knee or a hip. In another embodiment, the joint is a knee.

In an embodiment, the number and/or frequency of injections is lower than the number and/or frequency of injections with a corresponding viscosupplement comprising the hyaluronic acid and/or a derivative thereof in the physiologically acceptable buffered saline but devoid of the crystalline nanocellulose.

The present disclosure also includes a viscosupplement comprising:
hyaluronic acid and/or a derivative thereof; and
crystalline nanocellulose,
in a buffer solution.

It will be appreciated by a person skilled in the art that embodiments of the crystalline nanocellulose and the hyaluronic acid and/or a derivative thereof in such viscosupplements may be varied as described herein, for example, for the method of viscosupplementation and method of treating osteoarthritis of the present disclosure. It will also be appreciated by a person skilled in the art that in some embodiments the viscosupplements of the present disclosure are desirably physiologically acceptable. However, in other embodiments, such as for certain experimental testing, being physiologically acceptable may not be required. Accordingly, the buffer solution may comprise any suitable components that are optionally physiologically acceptable. It will be appreciated by a person skilled in the art that in viscosupplements, the solution would typically be an aqueous solution. The buffer solution may provide, for example, for maintaining the viscosupplement in a desired state, such as for example the ionic composition of the viscosupplement, the pH of the viscosupplement, the viscosity of the viscosupplement, or other desirable property for acceptable usage of the viscosupplement. The buffer solution may comprise a solution with an additional salt. The buffer solution may be selected from a physiologically acceptable buffer. Accordingly, in an embodiment, the buffer is a physiologically acceptable buffer. Non-limiting examples of the buffer solution include phosphate buffered saline (PBS) or another buffer solution which includes sodium chloride solution, such as physiological saline. In an embodiment, the buffer solution comprises a buffer and sodium chloride. In an embodiment, the buffer solution comprises a buffer and physiological saline. In an embodiment, the buffer solution is a physiologically acceptable buffered saline. In an embodiment, the physiologically acceptable buffered saline is a phosphate buffered saline (PBS). In an embodiment, the pH of the viscosupplement is 7.2±0.5. In another embodiment, the pH of the viscosupplement is 7.4+/−0.5.

In some embodiments of the present disclosure a viscoelastic composition is provided. In some embodiments of the present disclosure a viscosupplement composition is provided. In other embodiments of the present disclosure a viscosupplement for treatment of osteoarthritis is provided. In other embodiments of the present disclosure a method of treatment of osteoarthritis is provided. In still other embodiments of the present disclosure a method of viscosupplementation is provided. One aspect of the present disclosure provides for a viscoelastic (aqueous) mixture comprising a hyaluronic acid species or mixture of hyaluronic acid species, a crystalline nanocellulose species or a mixture of crystalline nanocellulose species, and a buffer solution. Another aspect of the present disclosure provides for a viscosupplement comprising a hyaluronic acid species or mixture of hyaluronic acid species, a crystalline nanocellulose species or a mixture of crystalline nanocellulose species, and a buffer solution. The present disclosure also includes a viscosupplement for the treatment of osteoarthritis comprising a mixture of hyaluronic acid species or mixture of hyaluronic species, a crystalline nanocellulose species or a mixture of crystalline nanocellulose species, and a buffer solution. The present disclosure also includes a viscosupplement for the treatment of osteoarthritis comprising a mixture of hyaluronic acid species or mixture of hyaluronic acid species, a crystalline nanocellulose species or a mixture of crystalline nanocellulose species, and a physiologically acceptable buffer. The present disclosure also includes a viscosupplement for the treatment of osteoarthritis comprising a mixture of hyaluronic acid species or mixture of hyaluronic acid species, a crystalline nanocellulose species or a mixture of crystalline nanocellulose species, and a physiologically acceptable buffer, the components of such viscosupplement present in any combination as disclosed herein. The present disclosure also includes a method of treating osteoarthritis, comprising injecting into the intra-articular space of a mammalian joint a viscosupplement comprising a mixture of hyaluronic acid species or mixture of hyaluronic acid species, a crystalline nanocellulose species or a mixture of crystalline nanocellulose species, and a physiologically acceptable buffer, the components of such viscosupplement present in any combination as disclosed herein. The present disclosure also includes a method of viscosupplementation of the synovial fluid of a mammalian joint, comprising injecting into the intra-articular space of the joint a viscosupplement comprising a mixture of hyaluronic acid species or mixture of hyaluronic acid species, a crystalline nanocellulose species or a mixture of crystalline nanocellulose species, and a physiologically acceptable buffer, the components of such viscosupplement present in any combination as disclosed herein.

The following are non-limiting examples of the present disclosure. These examples are also shown, for example, to demonstrate some of the enhanced properties of the viscosupplement provided by the present disclosure.

### Examples

For the following examples, the sodium salt of hyaluronic acid (HA) from Streptococcus equi (molecular weight 1.5-1.8 MDa) and phosphate buffered saline (PBS) powder were used. Freeze-dried sulfuric acid hydrolyzed nanocrystalline cellulose (CNC) powder was used. 1 g of PBS powder was mixed with 1 L of water using a hot plate magnet stirrer at 600 rpm for 1 hour to yield a PBS solution with a pH of 7.4. While continuing the stirring, the temperature of the mixture was raised to 37° C. While the temperature of the solution was maintained at 37±2° C. for 5 minutes, different concentrations of HA were added, and the stirring speed was increased to 1200 rpm and continued for another 11 hours. The suspension that formed after stirring was homogeneous and clear.

The CNC powder was then added at different weight percentages (for example ranging from 0.125% to more than 7%) and stirred using the magnetic stirrer for 6 hours, at a speed of 1200 rpm and at ambient temperature. For ease of reference herein, some of the samples discussed are numbered and named as shown in Table 1 below.

TABLE 1
Samples based on their HA and CNC concentrations.

| Sample | | HA concentration | CNC concentration |
|---|---|---|---|
| No. | Sample Name | (mg/ml) | (%) |
| 1 | HA | 1 | 0 |
| 2 | HA + 0.125CNC | 1 | 0.125 |
| 3 | HA + 0.25CNC | 1 | 0.25 |
| 4 | HA + 0.375CNC | 1 | 0.375 |
| 5 | HA + 0.5CNC | 1 | 0.5 |
| 6 | HA + 0.75CNC | 1 | 0.75 |
| 7 | HA + 1CNC | 1 | 1 |
| 8 | HA + 2CNC | 1 | 2 |
| 9 | HA + 3CNC | 1 | 3 |
| 10 | HA + 4CNC | 1 | 4 |
| 11 | HA + 5CNC | 1 | 5 |
| 12 | HA + 6CNC | 1 | 6 |
| 13 | HA + 7CNC | 1 | 7 |
| 14 | 0.5HA + 2CNC | 0.5 | 2 |
| 15 | 2HA + 2CNC | 2 | 2 |
| 16 | 3HA + 2CNC | 3 | 2 |
| 17 | 4HA + 2CNC | 4 | 2 |

In preparing the mixtures, various techniques may be used to improve mixing and solubilization of components, and to prepare stable mixtures. For example, mixtures may be prepared at selected temperatures including for example at temperatures ranging from 0° C. to 30° C. Furthermore, mixtures may be stirred and/or sonicated. For example, in some cases samples were sonicated with a probe sonicator (130 W and 20 kHz) at a 60% amplitude. A pulsatile sonication technique was used with pulse durations of 10 seconds to prevent damage to the HA and CNC from continuous sonication. The sonication treatment was performed in an ice bath to prevent thermal degradation of the CNCs. For example, vials containing 15 mL of HA+0.5CNC suspension were subjected to sonication treatment for 3, 6, 10, and 20 pulses with each pulse having a 10 second duration followed by a rest time of 10 seconds. After ultrasonication, the samples were allowed to stand, and the sedimentation was observed with a monochromatic light. The sample that had been subjected to 20 pulses showed minimal sedimentation and was stable for more than 2 hours.

To mimic the soft interface of cartilage in synovial joints, plates and balls made from poly dimethyl siloxane (PDMS) were used for the soft-tribology tests. The PDMS balls and plates were prepared by first mixing silicon elastomer base with a curing agent at a 10:1 ratio, respectively for about 15 minutes. The mixture was then degasified so that the resin was free of gas bubbles, which would otherwise affect the surface roughness of the final PDMS sheet and possibly allow the gas bubbles to migrate to the surface during the curing process. After degasification, the mixture was transferred to a mold to make the balls and to a petri dish to make the sheets and kept for 48 hours at room temperature for the curing process. After curing for 48 hours, the PDMS sheet and balls were removed from the petri dish and mold, rinsed, and treated with plasma to make the PDMS hydrophilic. The plasma treatment was carried out for 1 minute at a pressure of 0.7 Torr with a radio frequency (RF) power of 30 W (maximum RF power of the equipment used) in a plasma cleaner PDC-001 (Harrick Plasma). The PDMS sheets were cut into small squares and double-sided sticky nylon tape was used to fix them to the disc of the tribometer. The PDMS balls were inserted onto a specially made pin for the disc test.

Photomicrographs of the suspensions were taken with a Nikon Eclipse LV100N polarizing microscope. For the imaging, initially a 1 mL drop of each suspension was squeezed between two glass slides to form a thin film. The glass slides were then placed under the microscope to obtain photomicrographs of each sample. Under identical configurations, polarizing light was directed to each suspension to obtain the polarizing optical microscope (POM) images of the suspensions.

The steady shear viscosity of each sample was obtained using a Malvern Kinexus Ultra+Rheometer with a 2°/50 mm cone and plate measuring system. The steady shear viscosity of each sample was determined by varying the shear rates from 1000 s⁻¹ to 0.01 s⁻¹ at room temperature.

A Nanovea pin-on-disc tribometer was used to determine the coefficient of friction (COF) under different tribometry conditions. Three different tribo-pairs were used for the test, including steel-steel, ceramic-ceramic, and PDMS-PDMS. Since most joint prostheses work under a mixed lubrication regime, where the lubrication is influenced by the contact pair and the lubricant between the pair, in this study the loads and speeds for the pin-on-disc test were chosen for the tribological measurements were in mixed lubrication regime as shown in Table 2. The conditions were validated using empirical relationships for the mixed lubrication regime before the start of each test, and the tests were performed three times at room temperature.

TABLE 2
Pin on disc test conditions for the different friction pairs.

| Friction Pair | Load (N) | Speed (mm/min) |
|---|---|---|
| Steel-Steel | 5 | 150 |
| | 10 | 150 |
| Alumina ceramic-Alumina ceramic | 5 | 150 |
| | 10 | 150 |
| PDMS-PDMS | 2 | 75 |
| | 5 | 150 |

The steel-steel tribo-pairs were chosen to imitate a metal prosthetic knee. The radius of the circular path created by the pin was set to 5 mm and the duration of each test was set to 3 minutes. The tests were carried out for a shorter duration because of the tribo-chemical corrosion that was seen to occur between the steel ball and the HA that led to high fluctuations in COF when the test was performed for a long duration. By choosing a shorter duration, we could reduce the effect of corrosion on the COF and the wear characteristics. To understand the lubrication effect of samples when ceramics were used as the friction pair, the pin on disc test was performed with an alumina ceramic ball-alumina ceramic plate interface. The test durations were 1 hour and the wear path radius was set to 5 mm.

The soft-tribology tests using PDMS tribo-pairs were conducted with a wear path radius of 5 mm and a duration of 1 hr. The same procedure was followed as in the hard-tribology tests with the ceramic-ceramic friction pairs and steel-steel friction pairs.

For the λratio calculation for each tribology test, the minimum gap was calculated using the empirical relationships formulated by Hamrock and Dowson. The testing conditions can be classified into four different lubrication classes for the gap calculations as shown in Table 3. The calculations for determining the lubrication class were done iteratively, according to Hamrock and Dowson's method that accounts for the elastic deformation of the contact materials and the change in viscosity of the fluid samples due to the contact pressure during a test. The surface roughness of all surfaces in contact before the test were determined (both ball and disc for all three materials) with an optical profilometer. Using the minimum gap and roughness values, the lambda (λ) ratio was determined from Equation 1:

$$λ = \frac{{h}_{min}}{\sqrt{{( {R}_{q}^{pin} )}^{2} + {( {R}_{q}^{disc} )}^{2}}} \qquad ( 1 )$$

where R_{q}^{pin} is the root mean square (RMS) value of the roughness of the pin; R_{q}^{disc} is the RMS roughness value of the disc; and hₘᵢₙ is the minimum gap.

After the pin on disc tests, the plates were initially cleaned using acetone followed by sonication for 30 minutes in a sonication bath to remove any debris or impurities trapped on the wear track. After cleaning, the wear was characterized for all plates using a scanning electron microscope (SEM) and the surface topological characteristics were determined using an optical profilometer.

TABLE 3
Different lubrication classes based on the contact
pressure and the contact material property.

| | Influence of | Influence of | |
|---|---|---|---|
| | elastic | viscosity change | |
| | deformation of | due to the | |
| Class of | contact | contact | |
| lubrication | material | pressure | Conditions |
| Isoviscous-rigid | No influence | No influence | Very low contact pressure |
| Viscous-rigid | No influence | Influence | High contact pressure |
| | | | and contact materials |
| | | | with high Young's modulus |
| Isoviscous-elastic | Influence | No influence | Low contact pressure and |
| | | | contact materials with low |
| | | | elastic modulus or soft |
| | | | materials |
| Viscous-elastic | Influence | Influence | High contact pressure |

The surface features of the steel, ceramic, and PDMS plates after tribometry were observed using SEM. To make the PDMS and ceramic plates conductive and hence compatible for SEM imaging, the materials were initially sputter-coated. Iridium was used as the coating material. The conditions used for coating included an 80 mA current and a 10⁻² mbar pressure in the chamber with argon. After the condition was achieved, a 10 nm iridium coating was applied to the material surface. After the sputter coating, the edges of the plates were painted using a silver paint to create a clear conductive path to allow the charge to be easily grounded. Samples were kept aside for 24 hours for the curing process. The samples were mounted onto a small holder and the SEM images were taken for all three materials.

The topological features of the wear on the surface of the plates were evaluated experimentally using an optical profilometer with a 20× objective. To make the PDMS plates opaque, for observing the surface topology with the optical profilometer, a 10 nm thick iridium sputter-coating was applied to the PDMS surface before profilometric observations.

The wear characteristics were calculated using the root mean square value of the roughness (R_{q}) of the profile (using Equation 2) and the wear depth (Rₜ) was calculated (using Equation 3). The wear rate (\dot{V}) was calculated using Equation 4, which was derived with the assumption that the radius of the wear tract is the same as the radius of the path traced by the pin. For wear rate calculations, the wear area (A) at a particular location was calculated as the sum of the area subtended by two consecutive data points in the curve obtained from the profilometer (represented in Equation 5).

$${R}_{q} = \sqrt{\frac{{\sum }_{n = 1}^{N} ( {y}_{n} - \overline{y} )}{N}} \qquad ( 2 )$$

$${R}_{t} = {y}_{i} - {y}_{d} \qquad ( 3 )$$

$$\dot{V} = \frac{A \times 2 π r}{v \times t} \qquad ( 4 )$$

$$A = \int ydx \approx {\sum }_{n = 1}^{N - 1} ( ( {x}_{n + 1} - {x}_{n} ) \times ( {y}_{i} - {y}_{n + 1} ) + 1 / 2 \times ( {x}_{n + 1} - {x}_{n} ) \times ( {y}_{n + 1} - {y}_{n} ) ) \qquad ( 5 )$$

where xₙ is the x coordinate of the n^{th} point on the profile; xₙ₊₁ is the x coordinate of the (n+1)^{th} point on the profile; y̅ is the average of y coordinates of the roughness profile; yₙ is the y coordinate of the n^{th} point on the profile; yₙ₊₁ is the y coordinate of the (n+1)^{th} point on the profile; yᵢ is the y coordinate of the initial point on the profile (corresponding to the roughness of the initial plate before the test); y_{d} is the maximum depth of the profile (the lowest point in the profile); r is the radius of the circle traced by the pin (here it is 5 mm); ν is the speed of the pin; N is the total number of data points; and t is the duration of the tribological test.

To calculate different roughness parameters, the wear profiles at six different locations on the wear scar were taken and the mean of the data was reported.

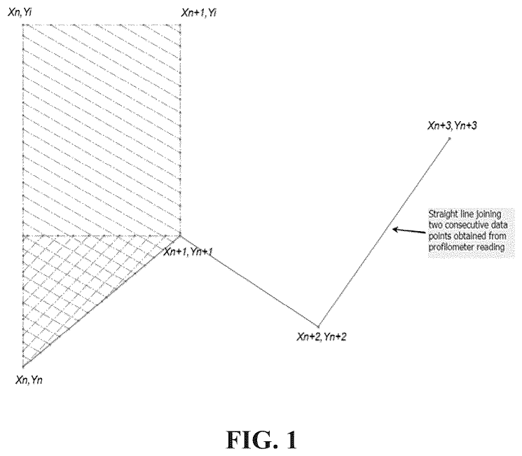

FIG. 1 shows a geometrical representation of the wear area calculation.

The rate of oxidation of the samples was studied with the in vitro, induced Weissberger's reaction using ascorbic acid and CuCl₂ reagents. For the reaction, a 10 mM CuCl₂ solution was first prepared by blending 0.27 g of CuCl₂ in 20 mL of distilled water. 1 μL of the 10 mM CuCl₂ solution was then pipetted into a 10 mL sample and stirred vigorously using a vortex stirrer for 30 seconds to yield the 1 μM CuCl₂ in suspension. The sample was kept for 7.5 minutes as a resting period. Likewise, 1.76 g of ascorbic acid was blended with 10 mL of distilled water to yield a 1 M ascorbic acid solution. Then, 1 μL of the 1 M ascorbic acid solution was pipetted into a 10 mL sample, which was then kept aside for a 7.5-minute resting period. Subsequently, the solution was stirred with a vortex stirrer to yield a final solution containing 1 μM CuCl₂ and 100 μM ascorbic acid in 10 mL of sample.

As shown in Scheme 1, the copper(II) ion from CuCl₂ and the ascorbic acid act as the oxidizing agent by producing H₂O₂ under aerobic conditions, which degrades HA by a free radical mechanism. The rate of reaction was assessed from the viscosity over 1.5 hours at a constant and optimized shear rate of 240 s⁻¹ using a rheometer with a 2°/50 mm cone and plate geometry. The rheometry tests began within 2 minutes after the ascorbic acid was added.

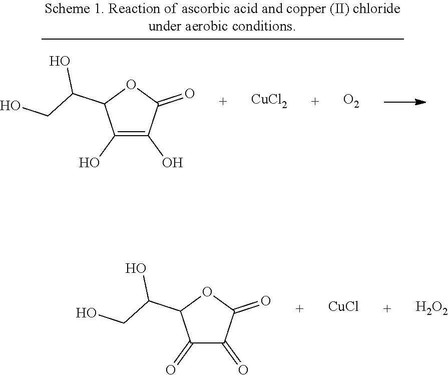

The percentage change in viscosity of the samples was calculated to assess the rate of oxidation using Equation 6:

$$\% change in viscosity = \frac{initial viscosity - final viscosity}{initial viscosity} \qquad ( 6 )$$

The addition of the CuCl₂ solution increased the apparent viscosity of the sample at a 240 s⁻¹ shear rate (testing condition) in comparison to the apparent viscosity of the sample without the CuCl₂ solution. Therefore, to neglect the effect of a slight increase in viscosity due to the addition of the CuCl₂ solution, for the percentage change in viscosity calculation, the initial viscosity value was taken from the viscometry data of raw samples without any reagents.

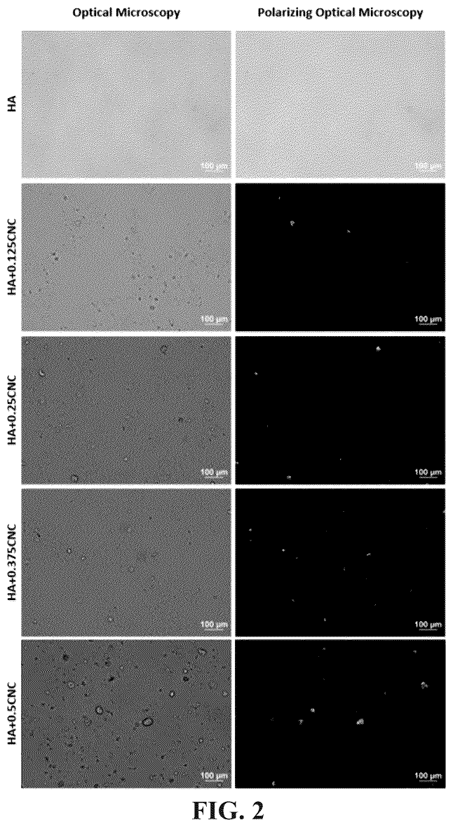

POM images and the photomicrographs of different samples under 500× zoom are shown in FIG. 2. The optically active structures observed in the POM images of CNC suspensions might be due to the self-assembly of CNC particles that form anisotropic chiral structures called tactoids. Since the tactoids were scattered throughout the sample at different planes, some tactoids were less bright or visible in the POM images. POM images and the photomicrographs of HA did not show any significant tactoids, but the photomicrographs of the samples with CNC showed clusters of some compounds mostly around the tactoids (FIG. 2). The clusters may have been the result of some intermolecular interactions between CNC and HA.

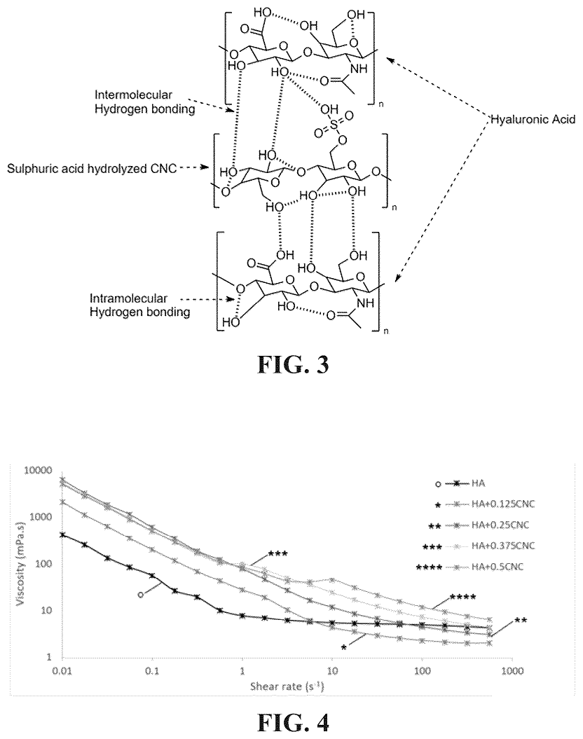

The predominant interaction occurring between HA and CNC is intermolecular hydrogen bonding, which arises from the presence of hydroxyl groups (—OH) and ether (—O—) groups in both CNC and HA, and carboxyl groups (—COOH) in HA and amide groups (—CONH—) in CNC. Some of these intermolecular hydrogen bonds are represented in FIG. 3.

The steady shear viscometry was studied at room temperature and selected results are shown in FIG. 4. All of these samples exhibited a shear thinning non-Newtonian behavior. At lower shear rates, the HA exhibited lower viscosity and as the CNC particles were added, the viscosity increased to a maximum value for HA+0.25CNC. Thereafter, the viscosity remained the same with increasing CNC concentration. At higher shear rates the HA viscosity exhibited a plateau-like behavior. These samples with CNC particles exhibited a significant shear thinning behavior even at high shear rates. Because of the plateau like trend occurred for HA at a high shear rate, the viscosity curves of two CNC suspensions with the lower concentrations (HA+0.125CNC and HA+0.25CNC) cross the viscosity curve of HA (FIG. 4). A small plateau was observed between two shear thinning behavior for HA+0.375CNC and HA+0.5CNC suspensions.

The shear thinning behavior can be explained by the randomness of the CNC particles at low shear rates that causes the higher viscosity. As the shear rate increases, the particles begin aligning along the direction flow, which reduces the viscosity. The increase in viscosity with the addition of CNC particles may be due to the agglomeration of molecules that occurs due to the strong intermolecular attraction between HA and CNC (as depicted in FIG. 3). The plateau that occurs between two shear thinning behavior for HA+0.375CNC and HA+0.5CNC suspensions may be due to the presence of anisotropic tactoids at higher concentrations.

The Herschel-Bulkley model (Equation 7) was used to fit the data obtained from the steady shear viscosity test. The fitted equation was used in the subsequent stages of this study:

$$τ = {τ}_{0} + m {\dot{γ}}^{n} \qquad ( 7 )$$

where τ is the shear stress; τ₀ is the yield stress; \dot{γ} the shear rate; m is the consistency parameter, and n is the constant exponent.

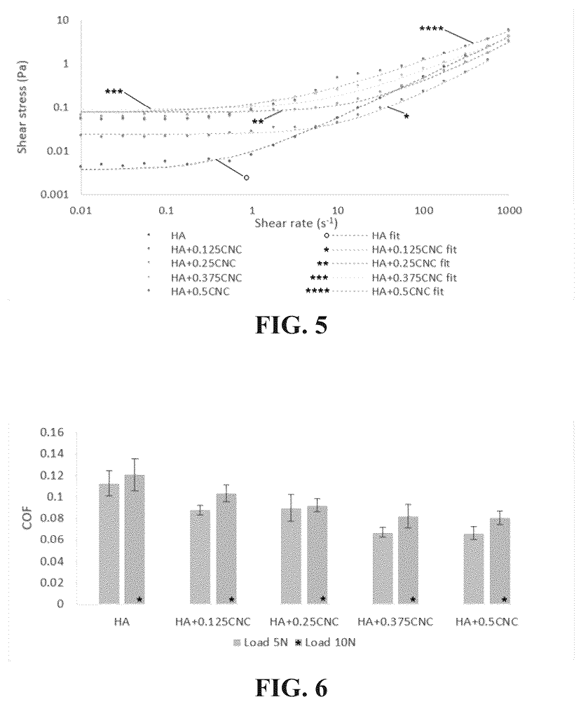

FIG. 5 represents the Herschel-Bulkley model curve fitting the steady shear viscometry data and Table 4 shows the model equation corresponding to each sample. The high R² value shows that the model equation fits with the experimental data and FIG. 5 supports the same conclusion as the curves from the experimental data almost coincide with the fitted equations.

TABLE 4
Herschel-Bulkley model equations for different samples.

| Sample | Herschel-Bulkley equation | R² |
|---|---|---|
| HA | τ = 0.00357 + 0.00626\dot{γ}^{0.93967} | 0.9999 |
| HA + 0.125CNC | τ = 0.02381 + 0.00197\dot{γ}^{0.9994} | 0.9996 |
| HA + 0.25CNC | τ = 0.07522 + 0.00384\dot{γ}^{0.96561} | 0.9980 |
| HA + 0.375CNC | τ = 0.08806 + 0.01498\dot{γ}^{0.80649} | 0.9973 |
| HA + 0.5CNC | τ = 0.07379 + 0.04429\dot{γ}^{0.69715} | 0.9970 |

The λratio was used to validate the mixed lubrication regime. When the λratio is less than 1, the system is in a thin film lubrication regime (boundary lubrication), an λratio between 1 and 3 indicates that a mixed lubrication regime is occurring, and an λratio greater than 3 indicates the presence of a hydrodynamic lubrication regime. Since the samples are non-Newtonian, the Herschel-Bulkley model equation was used for viscosity to determine the gap, which is then used to calculate the λratio. The modulus of elasticity and Poisson's ratio of the contact pairs used for the gap calculation are listed in Table 5. The class of lubrication, minimum gap, and λratio of different samples of steel, ceramic, and PDMS tribo-pairs, respectively under different testing conditions are shown in Tables 6-8.

TABLE 5
Modulus of Elasticity and Poisson's ratio for all tribo-pairs.

| Tribo-Pair | | Modulus of Elasticity (GPa) | Poisson's ratio |
|---|---|---|---|
| Steel | Ball | 200 | 0.300 |
| | Plate | 200 | 0.300 |
| Ceramic | Ball | 371 | 0.220 |
| | Plate | 304 | 0.210 |
| PDMS | Ball | 2.145 × 10⁻³ | 0.495 |
| | Plate | 2.145 × 10⁻³ | 0.495 |

TABLE 6
Class of lubrication, calculated minimum gap, and λ
ratios of the different samples for the steel-tribo pair.

| | | | Minimum | | | | Minimum | |
|---|---|---|---|---|---|---|---|---|
| Sample | | Class | Gap | λ ratio | | Class | Gap | λ ratio |
| HA | 5N | Viscoelastic | 2.05E−07 | 2.73 | 10N | Viscoelastic | 1.36E−07 | 1.81 |
| HA + 0.125CNC | 150 | | 1.38E−07 | 1.84 | 150 | | 9.29E−08 | 1.24 |
| HA + 0.25CNC | mm/min | | 1.74E−07 | 2.32 | mm/min | | 1.16E−07 | 1.56 |
| HA + 0.375CNC | | | 1.53E−07 | 2.05 | | | 9.75E−08 | 1.30 |
| HA + 0.5CNC | | | 1.56E−07 | 2.09 | | | 9.54E−08 | 1.27 |

TABLE 7
Class of lubrication, calculated minimum gap, and λ
ratios of different samples for the ceramic-tribo pair.

| | | | Minimum | | | | Minimum | |
|---|---|---|---|---|---|---|---|---|
| Sample | | Class | Gap | λ ratio | | Class | Gap | λ ratio |
| HA | 5N | Viscoelastic | 7.98E−07 | 2.53 | 10N | Viscoelastic | 5.3E−07 | 1.68 |
| HA + 0.125CNC | 150 | | 5.08E−07 | 1.61 | 150 | | 3.43E−07 | 1.09 |
| HA + 0.25CNC | mm/min | | 6.64E−07 | 2.11 | mm/min | | 4.43E−07 | 1.41 |
| HA + 0.375CNC | | | 6.92E−07 | 2.20 | | | 4.39E−07 | 1.39 |
| HA + 0.5CNC | | | 1.56E−07 | 2.09 | | | 4.94E−07 | 1.57 |

TABLE 8
Class of lubrication, calculated minimum gap, and λ ratios of different samples for the PDMS-tribo pair.

| | | | Minimum | | | | Minimum | |
|---|---|---|---|---|---|---|---|---|
| Sample | | Class | Gap | λ ratio | | Class | Gap | λ ratio |
| HA | 5N | Isoviscous- | 1.59E−08 | 2.64 | 2N | Isoviscous- | 1.24E−08 | 2.07 |
| HA + 0.125CNC | 150 | elastic | 1.19E−08 | 1.99 | 75 | elastic | 9.18E−09 | 1.53 |
| HA + 0.25CNC | mm/min | | 1.42E−08 | 2.36 | mm/min | | 1.1E−08 | 1.83 |
| HA + 0.375CNC | | | 9.26E−09 | 1.54 | | | 7.59E−09 | 1.26 |
| HA + 0.5CNC | | | 7.33E−09 | 1.22 | | | 6.28E−09 | 1.05 |

The iterative solving of the empirical relations revealed that both steel and ceramic materials were in the viscous-elastic class of lubrication, while PDMS was in the isoviscous-elastic lubrication class. High moduli of elasticity and the less deformable nature of steel and ceramic lead to the viscous-elastic classes for hard tribo-pairs, whereas PDMS was in the isoviscous-elastic class because of its very low modulus of elasticity and soft nature. The cartilage in human joints has been reported to be in the isoviscous-elastic lubrication class under normal conditions, which supports the idea that PDMS as the interface surface is a fair representation of soft nature of cartilage.

HA was observed to exhibit a higher gap, compared to other samples in the three tribo-pairs. During the tribology tests, the samples were subjected to very high shear rates and based on the steady viscometry test, HA had a higher viscosity illustrated by its plateau-like behavior, which led to a higher gap. In all cases shown, the λratio was between 1 and 3, showing that all selected conditions were under a mixed lubrication regime.

The COF of the samples was determined with a pin on disc apparatus using all three tribo-pairs. Each test was carried out three times and the average COF value with standard deviation is presented. FIG. 6 represents the COFs of selected samples under 5 N and 10 N loads and at a speed of 150 mm/min when steel was used as the tribo-pair. The COF for the HA sample was higher than that for the CNC suspensions and as the concentration of CNC particles in the suspension increased in these selected samples, the COF decreased under both loading conditions. The COF of all of these samples increased with an increase in load from 5 N to 10 N.

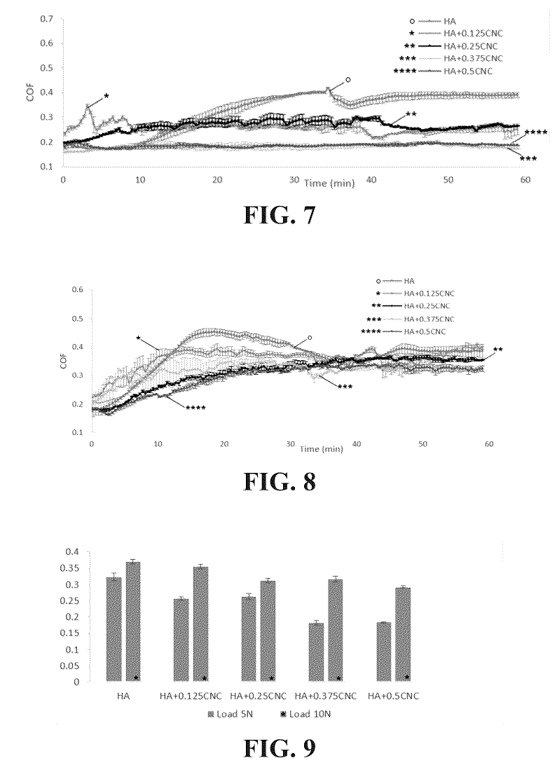

The tribology tests with the ceramic-tribo pairs showed the same trend as that for the steel-tribo pairs with increased COF values for the selected samples. The trend can be seen in the time evolution of COF represented in FIG. 7 and FIG. 8 under 5 N and 10 N loads, respectively, and in FIG. 9 that compares the COF of the samples at different loads for the ceramic interface.

For the tribology tests with hard friction pairs, the apparent viscosity can be considered constant for samples for all testing conditions since the speed (150 mm/min) and hence the shear rate remains the same. Therefore, from Equation 8 that represents the Hersey number, the only variable that affects the Hersey number is the normal load. Hence, as the load increases, the Hersey number decreases. According to the Stribeck curve, for a mixed lubrication regime, the COF must increase with a decrease in the Hersey number, which is supported by our results.

$$Hersey number = \frac{η {u}_{A}}{F} \qquad ( 8 )$$

where u_{A} is the velocity of the pin; F is the applied normal load; and η is the viscosity of the sample during the test, which remains constant throughout the test duration even for samples with shear dependent viscosity since the testing conditions (u_{A} and F) remain the same.

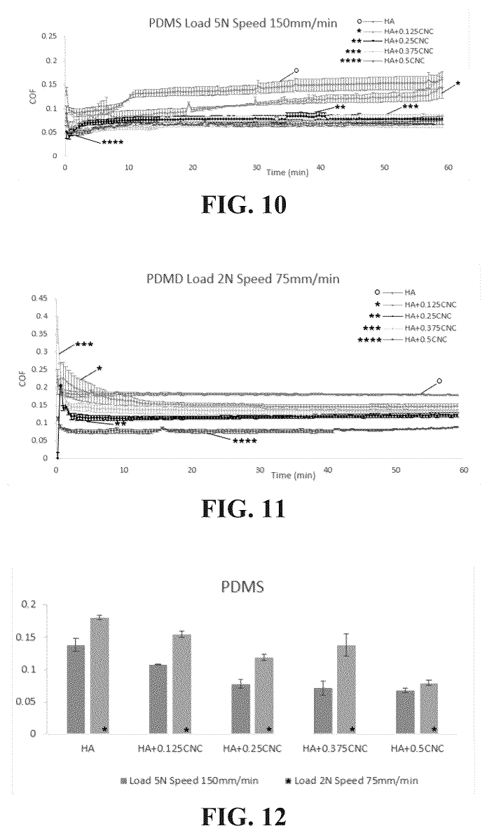

From the soft tribology tests with PDMS-tribo pairs, the COFs decreased with increasing CNC suspension concentrations. FIG. 10 and FIG. 11 represent the time-dependent COF under 5N load and 150 mm/min speed, and a 2N load and a 75 mm/min speed, respectively. Like the case of COFs for the hard interface tribology, HA+0.5CNC had the lowest COF values and HA had the highest COF value under all testing conditions. FIG. 12 shows that decreasing the speed and load from 150 mm/min and 5N (case 1) to 75 mm/min and 2N (case 2), increases the COF of all samples.

For the tribology test with PDMS friction pairs, the speed to load ratio remains the same as in case 1 and case 2. Hence, the only factor that affects the Hersey number is the viscosity of the sample. Since the PDMS is in the isoviscous-elastic class, the gap between the ball and plate is greatly influenced by the applied load. Hence as the load increases, the gap between the PDMS ball and the plate decreases, leading to an increased shear rate and decreased viscosity, assuming a shear-thinning behavior for the samples. Therefore, case 1 has a higher Hersey number than that of case 2. The Stribeck curve of a shear-thinning fluid with a soft interface will have two regions in the mixed lubrication regime named: 1^{st} transition region and 2^{nd} transition region. Since the lubrication regime is a mixed lubrication, a slight increase in COF with an increase in the Hersey number points to a second phase of the second transition region of the mixed lubrication regime under this condition.

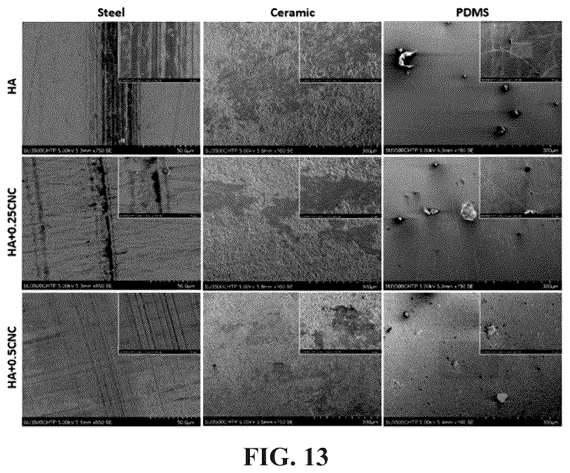

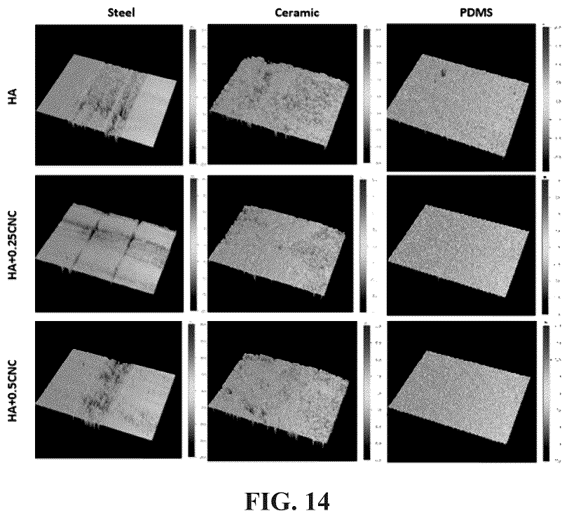

SEM images of wear scars on steel, ceramic and PDMS plates after the tribological tests (under a 5 N load and a 150 mm/min speed) are shown in FIG. 13. A clear wear tract can be seen that was generated in the alumina-ceramic plate and the steel plate, which was visible at low magnification, but a wear tract was not visible for the PDMS plate.

The tribology test with steel-tribo pairs showed a decrease in the wear on the plate after CNC particles had been added to HA. The surface topologies of the plates in all tribo-pair samples showed the most irregularities, when the tests were conducted with HA.

FIG. 14 shows wear scar diagrams of steel, ceramic, and PDMS plates from the optical profilometer after running the pin on disc test with a 5 N load and a 150 mm/min speed. HA had the highest wear scar compared to the other samples shown. The wear scar diagrams show the same trend as observed in the SEM images of the steel, ceramic, and PDMS plates.

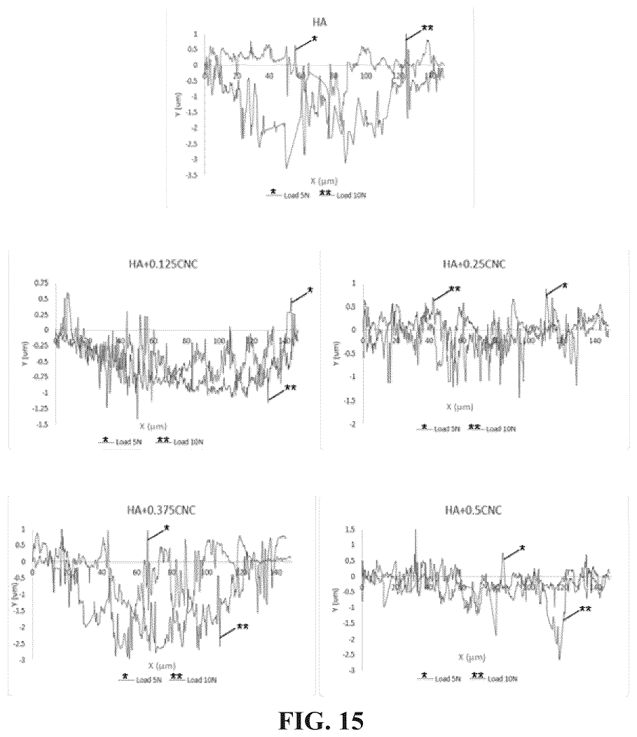

The roughness profiles of the steel plates, shown in FIG. 15, agree with the SEM images and the wear scar diagrams, except for the HA+0.5CNC sample. The highest surface roughness was observed for HA and it decreased when CNC particles were added. The roughness of the steel plate also increased with an increase in load from 5 N to 10 N for the samples.

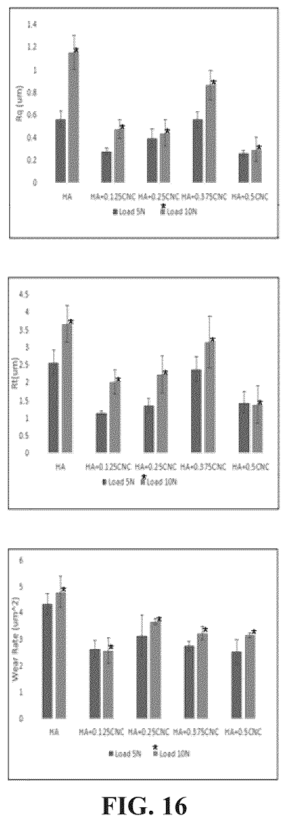

FIG. 16 shows, from top to bottom: the RMS values of roughness (R_{q}), wear depths (Rₜ) and wear rates, for the steel plates.

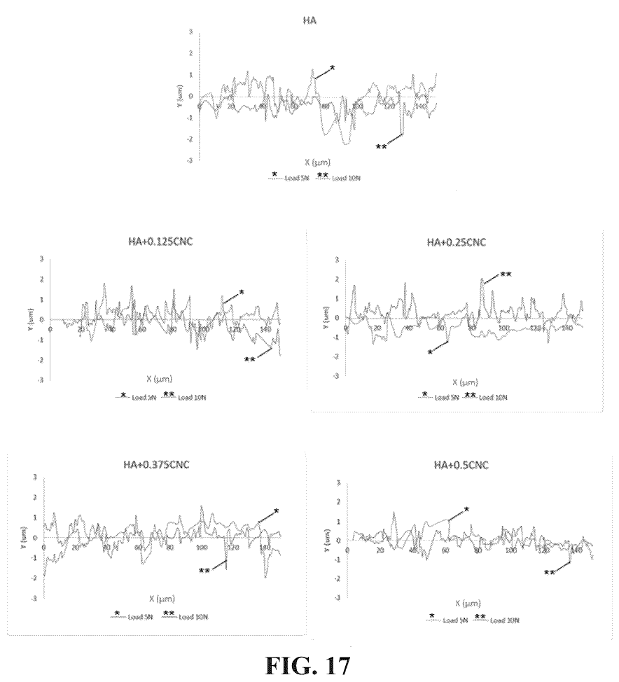

FIG. 17 shows the roughness profiles of the ceramic plates. The roughness profiles are in agreement with the trend depicted in the SEM images and the wear scar diagrams, with a decreasing roughness with an increasing concentration of CNC.

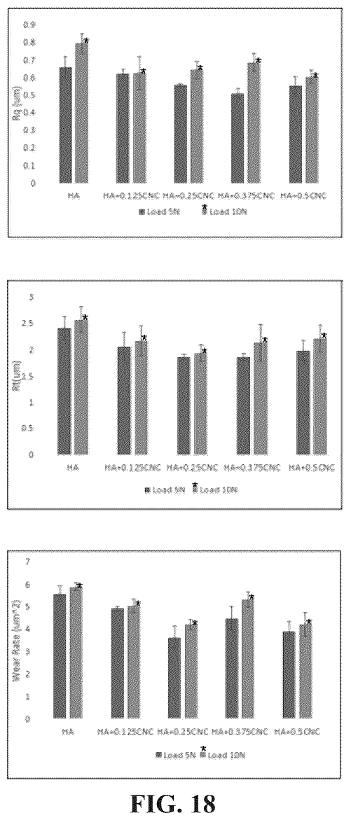

From the R_{q} for ceramic plates (FIG. 18, upper plot), under a 10 N load, the R_{q} was higher for HA and lower for HA+0.5CNC and the other samples showed almost the same range in values. Nevertheless, under a 5 N load, R_{q} showed a clear decreasing trend with increasing concentration of CNC, with a slight increase in R_{q} for the HA+0.5CNC sample. The Rₜ plot for ceramic plates (FIG. 18, middle plot) shows decreasing value with the concentration of CNC, with the lowest value for HA+0.25CNC. The values then increase with increasing concentrations of CNC. The wear rate in the bottom plot in FIG. 18 shows a similar trend to Rₜ with a slight decrease in the value for HA+0.5CNC.

The increase in wear with increasing loads for the steel and ceramic plates was due to the decrease in gap between the ball and plate. This led to a more direct contact and rubbing between the friction pairs with increased wear and greater surface roughness.

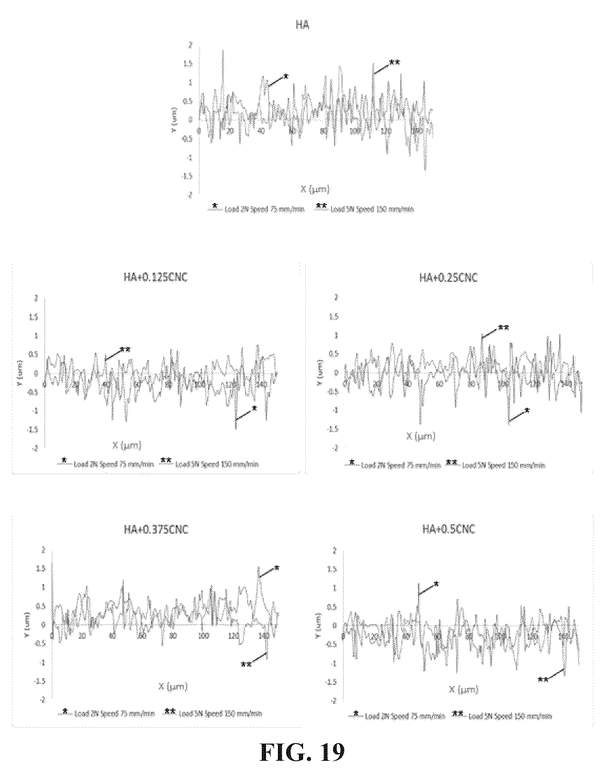

Like the roughness profiles of steel and ceramic, the PDMS plates also showed a higher value for HA than for the other samples (FIG. 19). The roughness profiles of all samples with CNC particles that were tested under the same condition were similar. Comparing the two testing conditions, the PDMS plates that were tested under a 5 N load and a 150 mm/min speed showed slightly more roughness than the roughness seen under the 2N load and 75 mm/min speed.

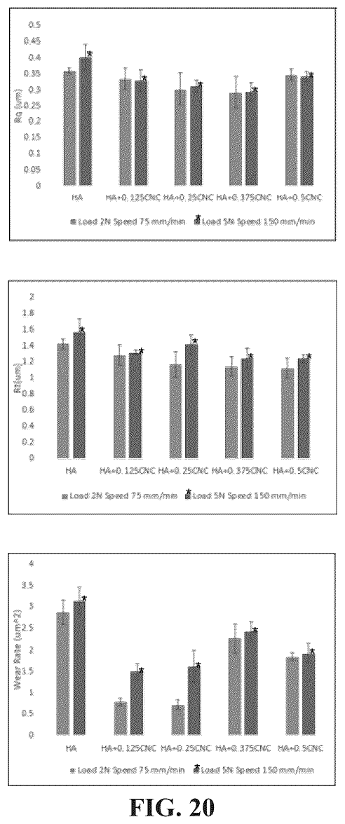

The R_{q}, Rₜ, and wear rates had the highest values for HA, compared to the other samples for PDMS (FIG. 20). Further, R_{q} and Rₜ had almost the same value for all of the CNC suspensions, but the wear rate followed a very different trend with the lowest value for HA+0.25CNC and HA+0.125CNC, under both testing conditions. The PDMS plates showed few topological changes, due to the soft nature of PDMS, which deforms easily and dissipates the friction energy by elastic deformation rather than by wear.

The decrease in COF and wear with the CNC concentration for all three friction pairs may be due to the mending effect and stick-slip behavior observed in CNC suspensions. CNC particles have a rod shape, and they can interact with the contact pair, forming a tribo-film over the surface. The tribo-film contributes to the reduced COF and reduced wear, by compensating for the unevenness in the surface, which increases the smoothness of the contact pair. This ability of CNC particles to form a tribo-film is called the mending effect. The stick-slip phenomenon is due to the ability of CNC particles to form intermolecular hydrogen bonding (H-bonding) with another CNC particle due to the presence of hydroxyl groups. The intermolecular interaction leads to the sticking and slipping of CNC particles over each other caused by the storage and release of elastic energy with the applied force. Because of the mending effect and CNC's ability to form H-bonds (stick behavior) the CNC particles form layers over the friction pair and can slip against each other, which helps reduce the COF and wear.

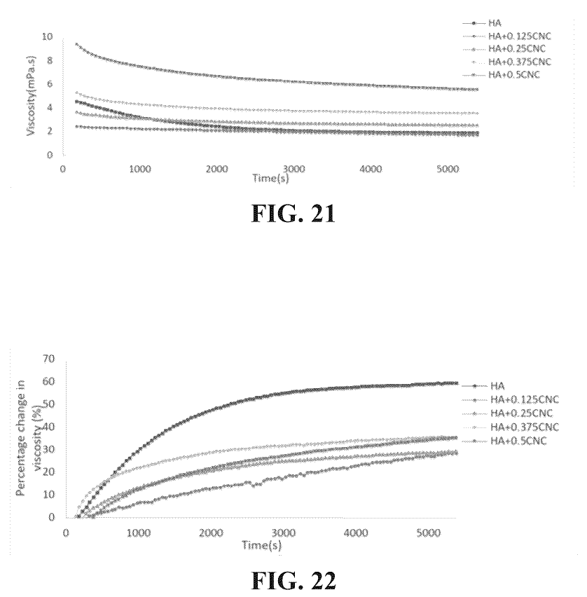

Weissberger's reaction was carried out and the viscosity of each sample after adding the oxidizing agent was recorded for 1.5 hours. (FIG. 21). After adding the oxidizing agent, the viscosity of samples began decreasing and the rate of decrease was initially steep and then followed by a slow rate. To compare the oxidation rate of different samples, the percentage change in viscosity with time was evaluated after adding the oxidizing agent to the samples (FIG. 22). The addition of CNC reduced the oxidative degradation rate of HA and as the concentration of CNC particles in the suspension increased, the resistance to degradation also increased, reaching lower degradation rates with the 0.125% and 0.25% CNC particle samples. HA showed a 59% change in viscosity, whereas HA+0.125CNC and HA+0.25CNC showed only a 29% change in viscosity. HA+0.375CNC and HA+0.5CNC had almost the same percentage change in viscosity (35%) at the end of the test.

Free radicals generated from the ROS, react with the HA and remove water through the homolytic cleavage of carbon-hydrogen bonds, producing HA with an alkyl free radical. The alkyl free radical undergoes a series of reactions to finally break the carbon-carbon bond and hence reduce the chain length that leads to the decrease in molecular weight of HA in the SF of OA patients. A decreased rate of degradation of HA after adding CNC was found. The free radical-scavenging capability of the CNC particles aids to reduce the number of free radicals generated by ROS.

Yet further examples are provided below, including where the CNC concentration ranges from 0.75-7% by weight are used, and the results are compared with a commercial viscosupplement that contains only Hylan.

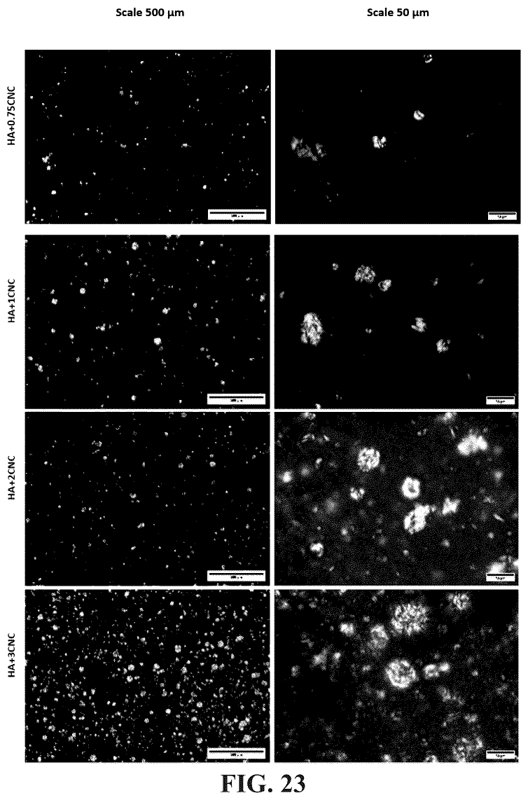

The polarizing optical microscopic images of samples is shown in FIG. 23.

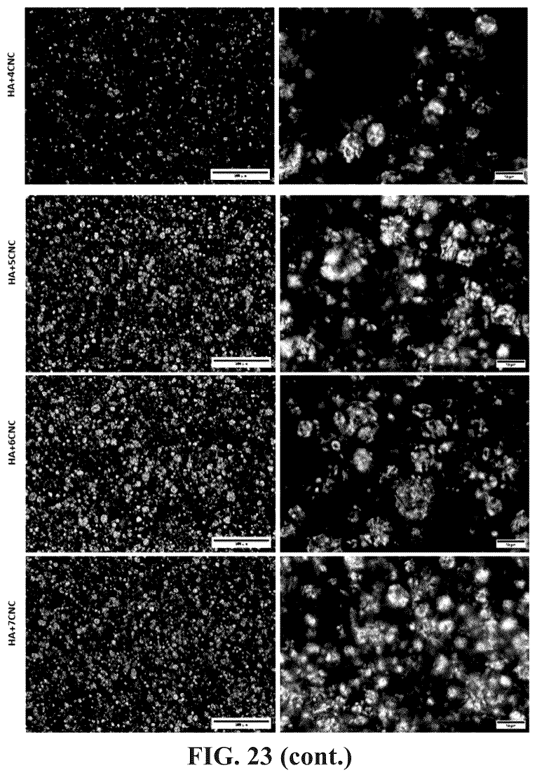

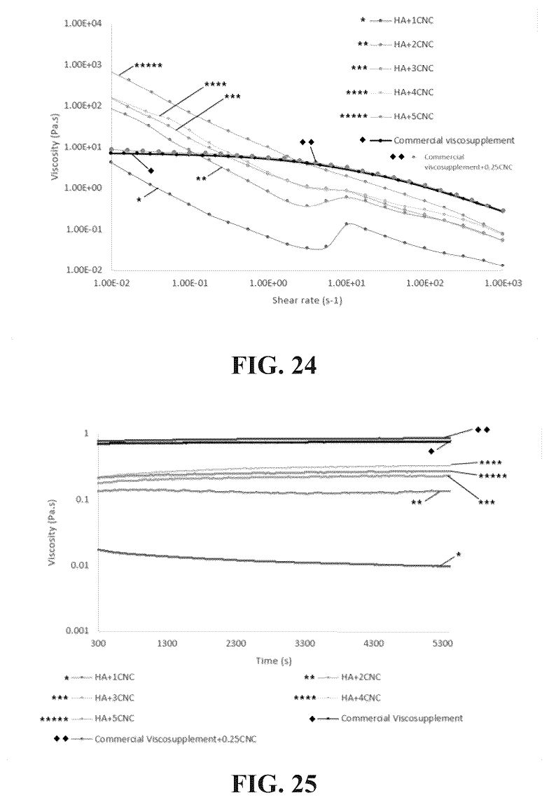

A steady shear viscometry test was conducted on samples by varying shear rate from 1000 s⁻¹ to 0.01 s⁻¹ and is represented in FIG. 24. All the HA/CNC samples were observed to be shear thinning with few samples with CNC concentrations ranging between 1-3% showing a bump/plateau in the intermediate shear rate that is attributed by the breakdown of agglomerates of CNC into smaller particles that leads to a frictional interaction between the particles. The commercial viscosupplement shows a constant viscosity trend at lower shear rate and a shear thinning nature at higher shear rates. Addition of 0.25% CNC to the commercial viscosupplement showed an increase in the viscosity.

The time dependent viscosity of the samples was determined at a constant shear rate of 240 s⁻¹ for 1.5 hrs. The time dependent viscosity of samples shows a rheopectic behavior for HA/CNC samples with concentration more than 2% which is represented in FIG. 25 and is the same trend as shown by healthy synovial fluid. The commercial viscosupplement also showed a small rheopectic behavior and the trend remains the same with the addition of 0.25% CNC with a slight increase in the viscosity value.

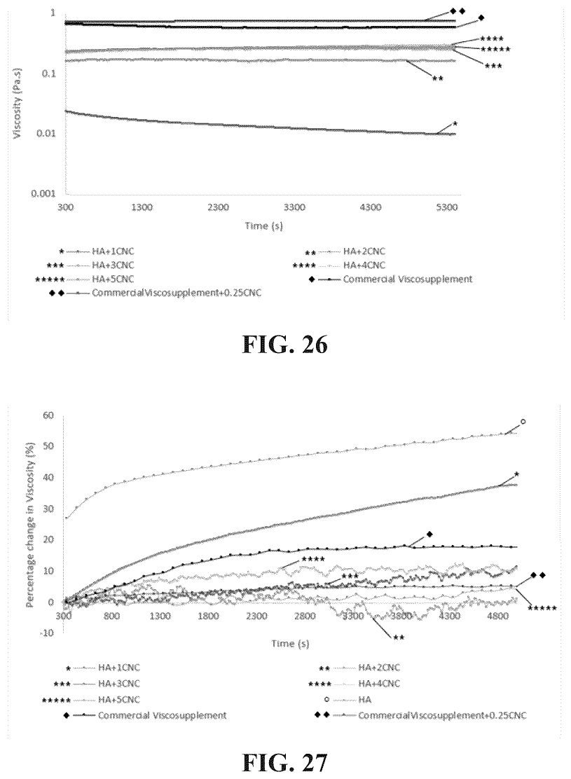

FIG. 26 represents time dependent viscosity of the samples after adding the oxidizing agent. Since time dependent viscosity of samples without adding oxidizing agents shows a rheopectic behavior, difference in slope of viscosity curve of samples with and without oxidizing agent was used to assess the rate of degradation.

The percentage change in viscosity (Equation 9) was used to evaluate the degradation.

$$\% change in viscosity = \frac{( μ ( t ) + k ) - {μ}_{oxidn} ( t )}{μ ( t ) + k} \qquad ( 9 )$$

where μ(t) is the viscosity of the sample without oxidizing agent at time t, μ_{oxidn}(t) is the viscosity of the sample with oxidizing agent at time t, and k is a factor that accounts for the instantaneous increase in viscosity due to the addition of copper chloride to the oxidizing sample.

$$k = {μ}_{oxidn} ( {t}_{0} ) - μ ( {t}_{0} ) \qquad ( 10 )$$

where t₀ is the starting time of analysis, here it t₀=300 s is used to account for inertia.

FIG. 27 that represents percentage change in viscosity indicates that the rate of degradation decreases by considerably amount after the addition of CNC over 2%. After 1.5 hours the percentage change in viscosity of samples with more than 2% CNC is less than 10%. Oxidative study of HA indicates a percentage change in viscosity of over 50% for hyaluronic acid after 1.5 hr. The commercial viscosity without CNC indicates an oxidative degradation of 20% after 1.5 hours decreased to 5% after adding only 0.25% CNC.

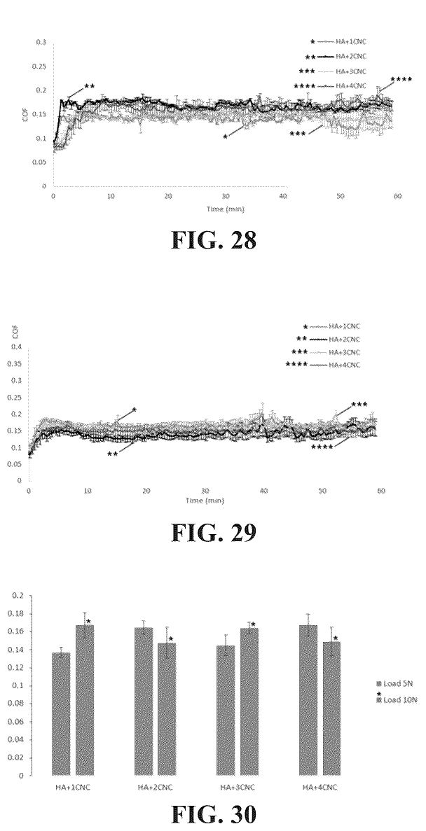

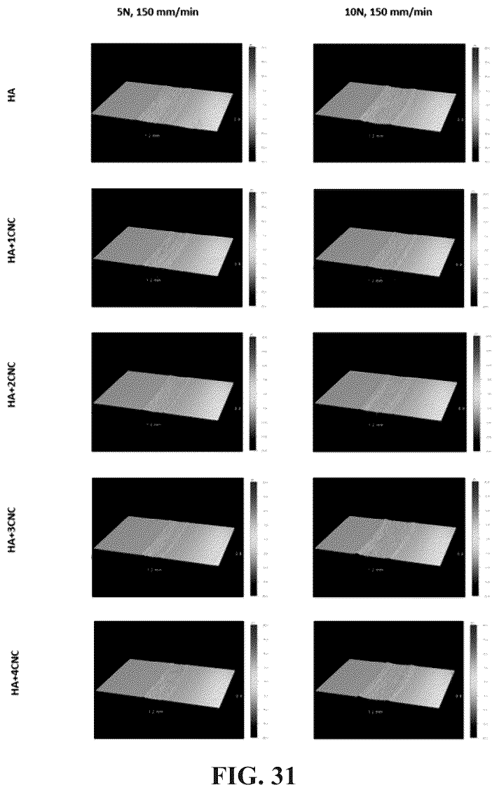

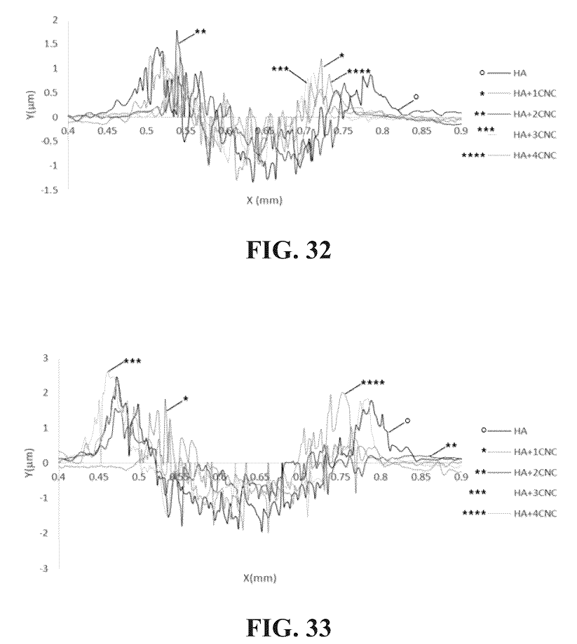

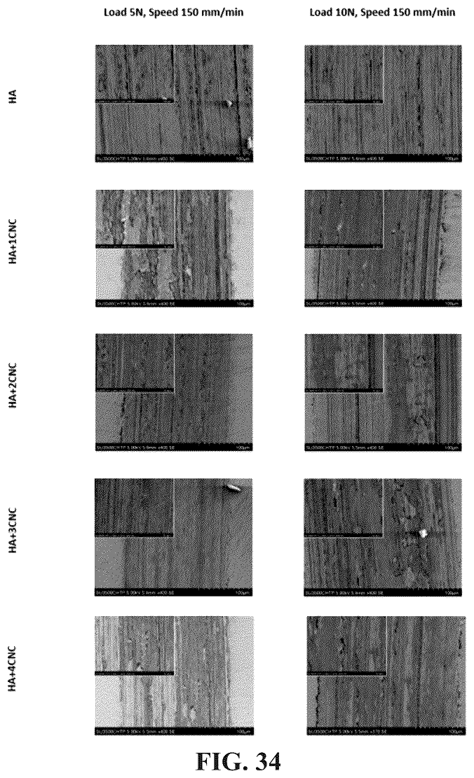

The tribological characterization of the samples was performed on a Nanovea pin on disc tribometer with steel-steel friction pair at loads of 5 N and 10 N and speed of 150 mm/min. The time dependent Coefficient of friction (COF) (FIG. 28 and FIG. 29) indicates that the COF value attains a steady state value for all the samples before 10 minutes. The COF values of the samples and loads were in the same range (FIG. 30) and was lower than the COF value observed for HA sample. To generate the wear tract for HA sample after running the test for 1 hour the pin on disc test was carried out for HA along with other samples with the same testing conditions. From FIG. 31 which represents the 3D profile of the wear tract, it can be observed that the wear is more in HA than samples with CNC. FIG. 32 and FIG. 33 show the roughness profile of the wear tract generated during pin on disc test at 150 mm/min speed and 5N and 10 N load respectively. While analyzing the roughness of the wear, a clear distinction between different samples cannot be drawn. However, it was observed from scanning electron microscopy (SEM) images (FIG. 34) that the steel plate has highest surface irregularity when HA is used as the lubricant as compared to other samples as the CNC helps in wear reduction.

Yet further examples are provided below, including where the CNC concentrations are kept constant at 2% and different HA concentrations including 0.5 mg/ml, 2 mg/ml, 3 mg/ml and 4 mg/ml were used.

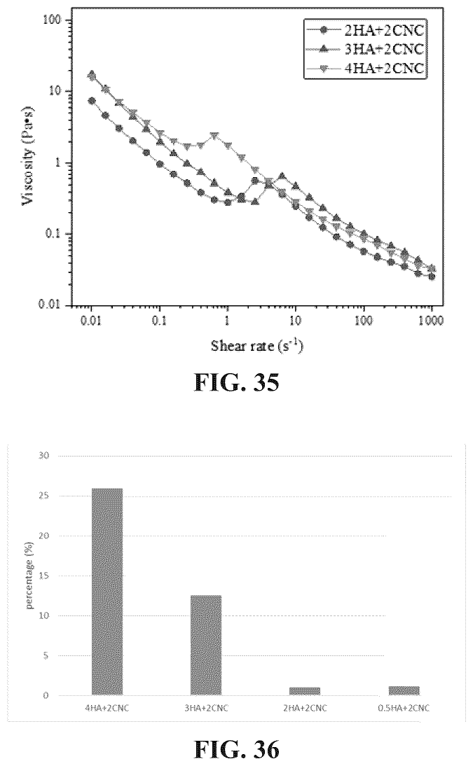

Steady shear viscometry test for the HA/CNC suspension samples at 2% CNC and varying HA concentrations was conducted and the results shown in FIG. 35. All the samples showed a shear thinning behavior with a hump at intermediate shear rate due to the breakdown of agglomerates of CNC and frictional interaction between the particles. It was also observed that the low shear viscosity generally showed an increasing trend with the increase in HA concentration. The percentage change in viscosity was measured after 1.5 hours. after adding the oxidizing agent into the HA/CNC suspensions containing 2% CNC and varying HA concentrations is shown in FIG. 36. It can be observed that at higher concentration of HA the oxidative degradation is very high whereas at lower concentrations it is as low as 2%.

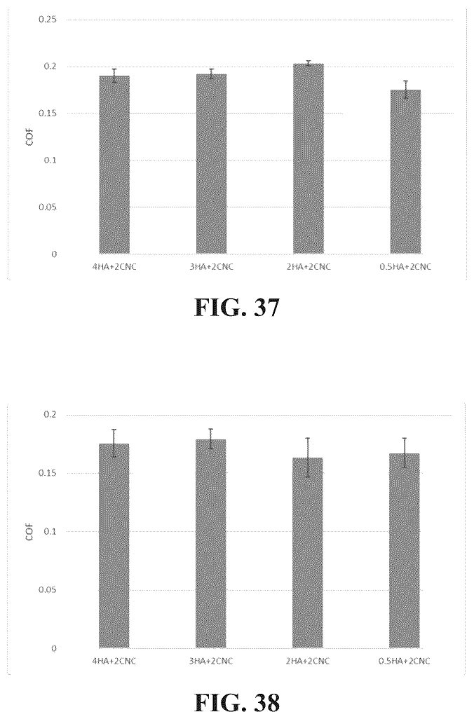

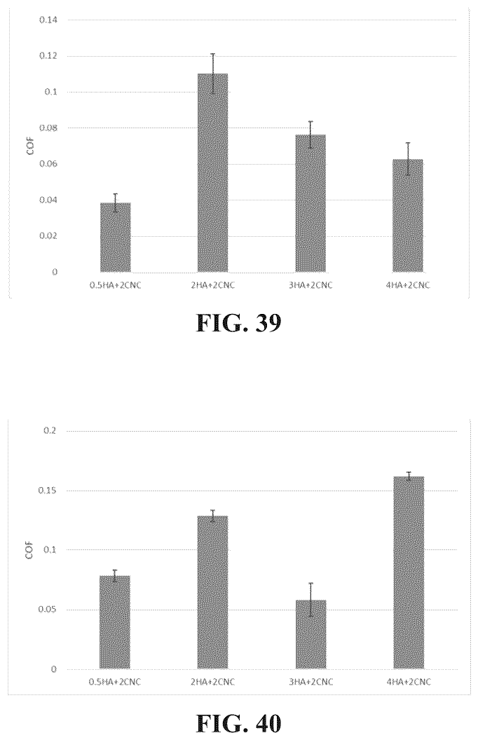

The coefficient of friction of the samples with 2% CNC and 0.5, 2, 3 and 4 mg/ml HA was measured using a pin on disc tribometer using a steel friction pair under the speed of 150 mm/min and loads of 5 N and 10 N and the results shown in FIG. 37 and FIG. 38 respectively. It was observed that varying the HA concentrations had no significant effect on the coefficient of friction when steel was used as the friction pair. The coefficient of friction of the samples with varying HA concentration at a constant CNC concentration of 2% with PDMS friction pair under 150 mm/min speed and loads of 2 N and 5 N is shown in FIG. 39 and FIG. 40 respectively. It was observed that the 0.5HA+2CNC had the lowest COF among all the samples when PDMS was used. 3HA+2CNC also showed a lower COF value.

As shown in the examples provided herein, the addition of CNC to HA reduces the wear and COF and provide compositions that show a high degree of resistance to oxidation, especially when compared to HA without the added CNC. The improved tribological performance and resistance to oxidation make such CNC particle suspensions in HA a better lubricant with longer lasting effects, compared to HA alone, and can provide enhanced performance for viscosupplements, including for the treatment of osteoarthritis.

While the present disclosure has been described with reference to examples, it is to be understood that the scope of the claims should not be limited by the embodiments set forth in the examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

1. A method of viscosupplementation of the synovial fluid of a joint in a mammalian subject, the method comprising:
intra-articularly injecting into the joint, a viscosupplement comprising:
hyaluronic acid and/or a derivative thereof; and
crystalline nanocellulose,
in a physiologically acceptable buffered saline.

2. The method of claim 1, wherein the viscosupplementation is for treatment of arthritis.

3. The method of claim 2, wherein the arthritis is osteoarthritis.

4. The method of claim 1, wherein the crystalline nanocellulose has been prepared by a method comprising sulphuric acid hydrolysis.

5. The method of claim 4, wherein the crystalline nanocellulose is present at a concentration of from 0.1 wt. % to 3 wt. %, based on the total weight of the viscosupplement.

6. The method of claim 1, wherein the hyaluronic acid and/or derivative thereof comprises a sodium salt of hyaluronic acid.

7. The method of claim 6, wherein the hyaluronic acid is from a bacterial source.

8. The method of claim 1, wherein the physiologically acceptable buffered saline is a phosphate-buffered saline.

9. The method of claim 1, wherein the mammalian subject is a human.

10. The method of claim 1, wherein the mammalian subject is a dog or a horse.

11. The method of claim 1, wherein the number and/or frequency of injections is lower than the number and/or frequency of injections with a corresponding viscosupplement comprising the hyaluronic acid and/or a derivative thereof in the physiologically acceptable buffered saline but devoid of the crystalline nanocellulose.

12. A viscosupplement comprising:
hyaluronic acid and/or a derivative thereof; and
crystalline nanocellulose,
in a buffer solution.

13. A method of treating osteoarthritis in a joint of a mammalian subject suffering from osteoarthritis in the joint, the method comprising:
intra-articularly injecting into the joint, a viscosupplement comprising:
hyaluronic acid and/or a derivative thereof; and
crystalline nanocellulose,
in a physiologically acceptable buffered saline.

14. The method of claim 13, wherein the crystalline nanocellulose has been prepared by a method comprising sulphuric acid hydrolysis and/or wherein the crystalline nanocellulose is present at a concentration of from 0.1 wt. % to 3 wt. %, based on the total weight of the viscosupplement.

15. The method of claim 13, wherein the hyaluronic acid and/or derivative thereof comprises a sodium salt of hyaluronic acid.

16. The method of claim 15, wherein the hyaluronic acid is from a bacterial source.

17. The method of claim 13, wherein the physiologically acceptable buffered saline is a phosphate-buffered saline.

18. The method of claim 13, wherein the mammalian subject is a human.

19. The method of claim 13, wherein the mammalian subject is a dog or a horse.

20. The method of claim 13, wherein the number and/or frequency of injections is lower than the number and/or frequency of injections with a corresponding viscosupplement comprising the hyaluronic acid and/or a derivative thereof in the physiologically acceptable buffered saline but devoid of the crystalline nanocellulose.
